(19) United States
(12) Patent Application Publication
(10) Pub. No.: US 2019/0000428 A1
(43) Pub. Date: Jan. 3, 2019

(54) ASPIRATION FILTRATION DEVICE

(71) Applicant: BOSTON SCIENTIFIC SCIMED, INC., Maple Grove, MN (US)

(72) Inventors: JEFF GRAY, Sudbury, MA (US); SERENA SCOTT, Worcester, MA (US); SHANNON SMITH KENWOOD, Hopkinton, MA (US); GENE T. STORBECK, Franklin, MA (US)

(73) Assignee: BOSTON SCIENTIFIC SCIMED, INC., Maple Grove, MN (US)

(21) Appl. No.: 16/023,274

(22) Filed: Jun. 29, 2018

Related U.S. Application Data
(60) Provisional application No. 62/527,666, filed on Jun. 30, 2017.

(51) Int. Cl. A61B 10/02; A61M 1/00

(52) U.S. Cl. CPC A61B 10/0283; A61B 2010/045; A61B 10/0266; A61M 1/0056

(57) ABSTRACT

A tissue filtration device may include an elongate hollow needle assembly having open distal and proximal ends. In some cases, the tissue filtration device may include a collection device containing a filter material and coupled to the elongate hollow needle assembly and in fluid communication with the elongate hollow needle. Also included may be a suction assembly coupled to the collection device and configured to draw fluid from the elongate hollow needle assembly into the collection device.

### CROSS-REFERENCE TO RELATED APPLICATIONS

This application claims priority under 35 U.S.C. § 119 to U.S. Provisional Application Ser. No. 62/527,666, filed Jun. 30, 3017, the entirety of which is incorporated herein by reference.

### TECHNICAL FIELD

The present disclosure pertains to fine needle aspiration (FNA) devices, and methods for manufacturing and/or using FNA devices. More particularly, the present disclosure pertains to filtration devices for FNA devices and methods, and operation and methods for such devices.

### BACKGROUND

A wide variety of medical devices have been developed for medical use, for example for collecting and/or processing biological samples. Some of these devices include filtration devices. Of the known medical devices and methods, each has certain advantages and disadvantages. There is an ongoing need to provide alternative medical devices as well as alternative methods for using medical devices.

### BRIEF SUMMARY

This disclosure provides design, material, manufacturing method, and use alternatives for medical devices, including delivery devices.

In a first example, a tissue filtration device may comprise a distal elongate hollow needle assembly defining a needle lumen and having open distal and proximal ends, a medial collection device in fluid communication with the needle lumen and containing a filter material, and a proximal suction assembly configured to draw fluid from the needle lumen into the medial collection device.

Alternatively or additionally to any of the examples above, in another example, the distal elongate hollow needle assembly may include an elongate hollow needle and a hollow tube proximal of the elongate hollow needle.

Alternatively or additionally to any of the examples above, in another example, the proximal suction assembly may include a handle assembly and a hollow tube distal of the handle assembly.

Alternatively or additionally to any of the examples above, in another example, the handle assembly may include a piston inside a cylindrical tube and the fluid is drawn from the needle lumen of the distal elongate hollow needle to the medial collection device by moving the piston through the cylindrical tube.

Alternatively or additionally to any of the examples above, in another example, the handle assembly may include a mechanism and the fluid is drawn from the needle lumen of the distal elongate hollow needle assembly to the medial collection device by actuating the mechanism.

Alternatively or additionally to any of the examples above, in another example, the filter material may filter tissue from the fluid.

Alternatively or additionally to any of the examples above, in another example, the filter material may include a filter membrane.

Alternatively or additionally to any of the examples above, in another example, the medial collection device may comprise an exterior casing configured to maintain an airtight seal when punctured by the distal elongate hollow needle assembly.

Alternatively or additionally to any of the examples above, in another example, the exterior casing may comprise a plastic.

Alternatively or additionally to any of the examples above, in another example, the filter material may filter cells over about 0.1 to 10 μm[ssi] in diameter.

In another example, a fine needle aspiration (FNA) device may comprise a distal elongate hollow needle assembly defining a needle lumen and having open distal and proximal ends, a medial collection device in fluid communication with the needle lumen and containing a filter material, and a proximal handle assembly configured to draw fluid from the needle into the collection device.

Alternatively or additionally to any of the examples above, in another example, the medial collection device may be coupled to the distal elongate hollow needle assembly using a connecting mechanism.

Alternatively or additionally to any of the examples above, in another example, the proximal handle assembly may be coupled to the collection device using a connecting mechanism.

Alternatively or additionally to any of the examples above, in another example, the distal elongate hollow needle assembly may include an elongate hollow needle and a hollow tube proximal of the elongate hollow needle.

Alternatively or additionally to any of the examples above, in another example, the proximal handle assembly may include a handle and a hollow tube distal of the handle.

Alternatively or additionally to any of the examples above, in another example, the handle includes a piston inside a cylindrical tube and the fluid is drawn from the needle lumen into the medial collection device by moving the piston through the cylindrical tube.

Alternatively or additionally to any of the examples above, in another example, the handle includes a mechanism and the fluid is drawn from the needle lumen into the medial collection device by actuating the mechanism.

Alternatively or additionally to any of the examples above, in another example, the filter material may filter tissue from the fluid.

Alternatively or additionally to any of the examples above, in another example, the filter material may include a filter membrane.

In some examples, a collection device may be configured to receive fluid from a biopsy needle, the collection device may comprise an exterior casing configured to maintain an airtight seal when punctured by the biopsy needle, a filter membrane substantially enclosed by the exterior casing and configured to filter tissue from the fluid, and an opening configured to couple the collection device to a suction assembly enabling the suction assembly to draw the fluid from the biopsy needle into the collection device.

This summary is intended to provide an introduction to the subject matter of the present patent application. It is not intended to provide an exclusive or exhaustive explanation of the invention. The detailed description is included to provide further information about the present patent application.

### BRIEF DESCRIPTION OF THE DRAWINGS

The disclosure may be more completely understood in consideration of the following detailed description in connection with the accompanying drawings, in which:

FIGS. 1A-1B are views of an illustrative tissue filtration device;

FIG. 1C is a view of the operation of the illustrative tissue filtration device;

FIG. 2A is a view of cross-flow filtration;

FIG. 2B is a view of dead-end filtration;

FIG. 3A is a view of a second illustrative tissue filtration device;

FIG. 3B is a view of the operation of the second illustrative tissue filtration device;

FIGS. 4A-4B are views of tissue filtration system;

FIGS. 5A-5B are views of a second tissue filtration system;

FIGS. 6A-6E are views of a third tissue filtration system; and

FIG. 7 is a view of an exemplary flow-method.

While the disclosure is amenable to various modifications and alternative forms, specifics thereof have been shown by way of example in the drawings and will be described in detail. It should be understood, however, that the intention is not to limit the invention to the particular embodiments described. On the contrary, the intention is to cover all modifications, equivalents, and alternatives falling within the spirit and scope of the disclosure.

### DETAILED DESCRIPTION

For the following defined terms, these definitions shall be applied, unless a different definition is given in the claims or elsewhere in this specification.

All numeric values are herein assumed to be modified by the term “about,” whether or not explicitly indicated. The term “about” generally refers to a range of numbers that one of skill in the art would consider equivalent to the recited value (i.e., having the same function or result). In many instances, the terms “about” may include numbers that are rounded to the nearest significant figure.

The recitation of numerical ranges by endpoints includes all numbers within that range (e.g. 1 to 5 includes 1, 1.5, 2, 2.75, 3, 3.80, 4, and 5).

As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise.

It is noted that references in the specification to “an embodiment”, “some embodiments”, “other embodiments”, etc., indicate that the embodiment described may include one or more particular features, structures, and/or characteristics. However, such recitations do not necessarily mean that all embodiments include the particular features, structures, and/or characteristics. Additionally, when particular features, structures, and/or characteristics are described in connection with one embodiment, it should be understood that such features, structures, and/or characteristics may also be used connection with other embodiments whether or not explicitly described unless clearly stated to the contrary.

The following detailed description should be read with reference to the drawings in which similar structures in different drawings are numbered the same. The drawings, which are not necessarily to scale, depict illustrative embodiments and are not intended to limit the scope of the disclosure.

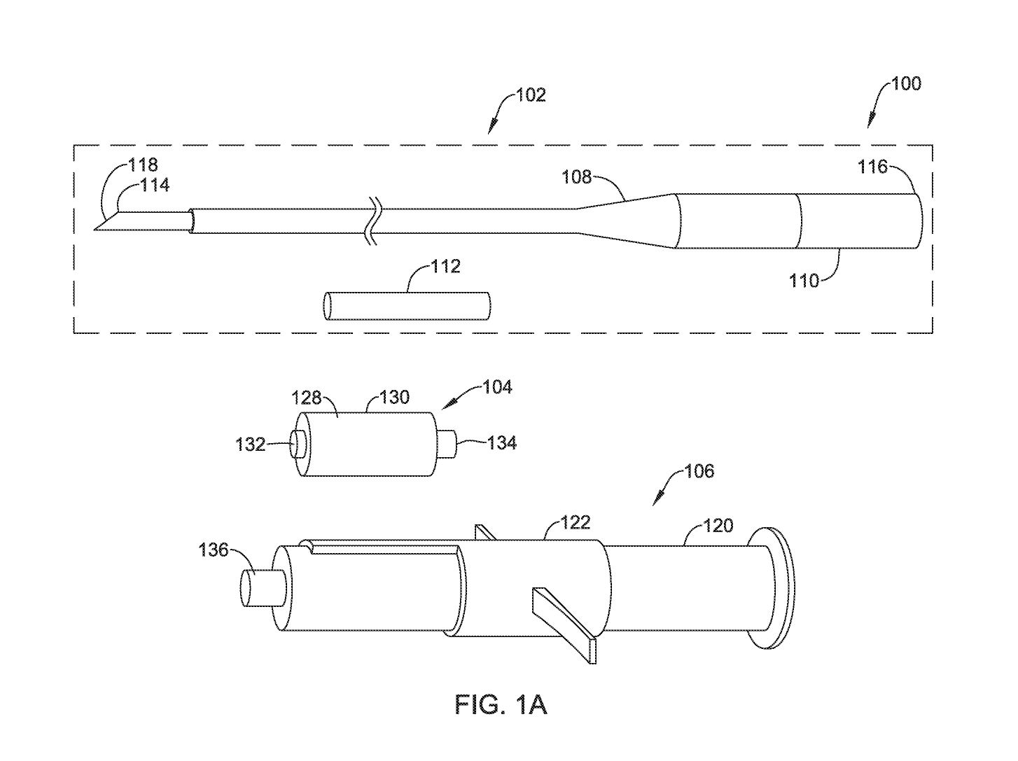

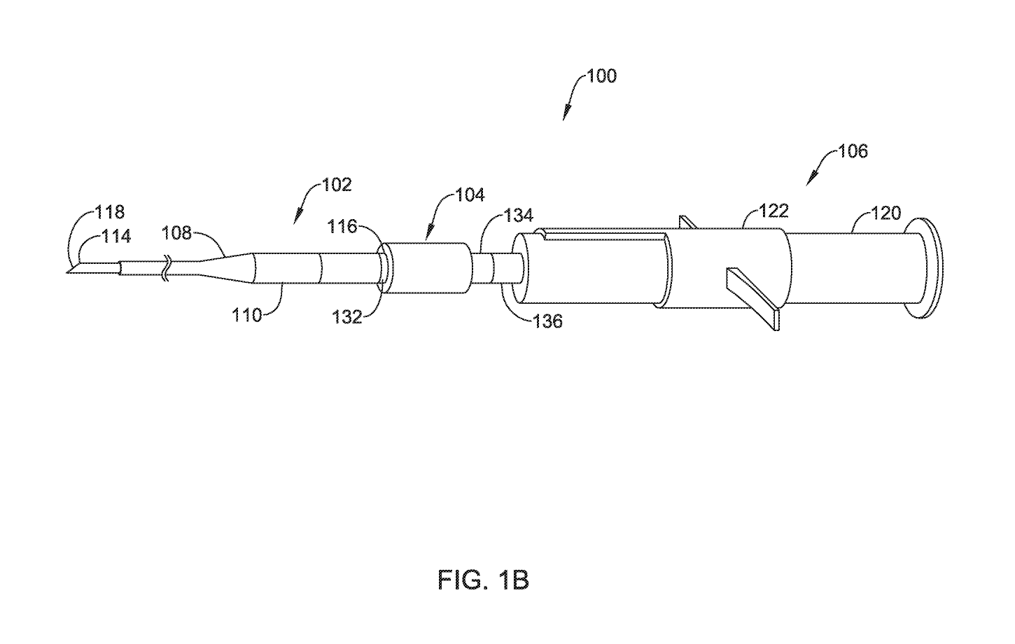

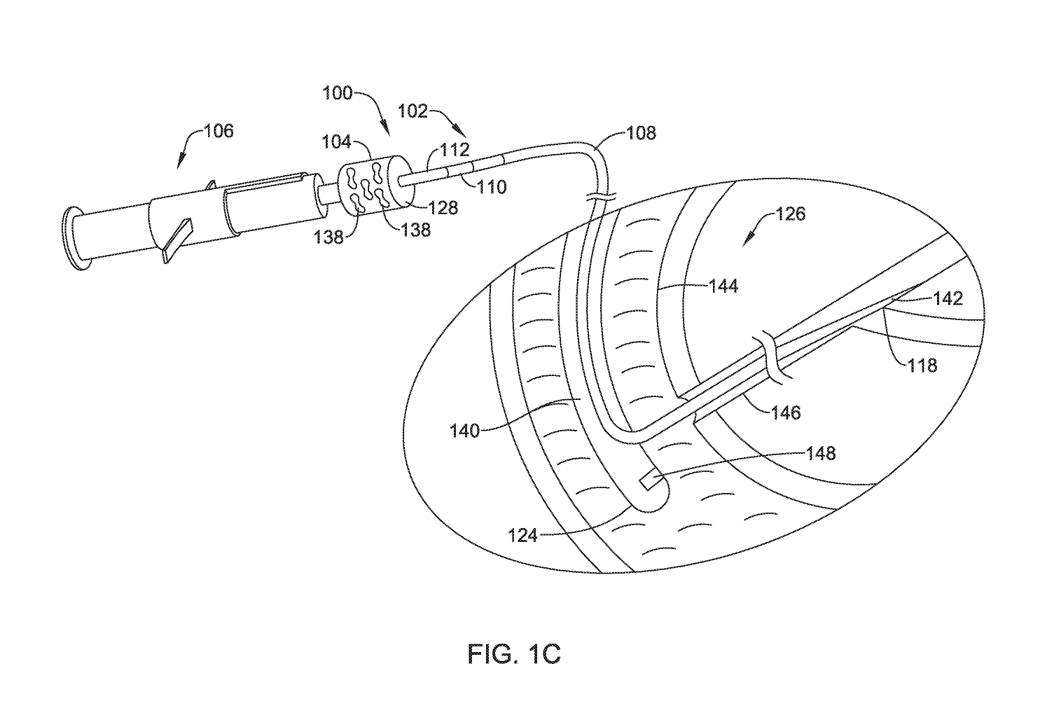

FIG. 1A illustrates an example of a disassembled tissue filtration device 100 that may be used to aspirate fluid from a patient and separate tissue samples from the fluid. FIG. 1B illustrates the assembled tissue filtration device 100. According to various embodiments, the tissue filtration device 100 may include an elongate hollow needle assembly 102, a collection device 104, and a suction assembly 106. Fine-needle aspiration (FNA) is a diagnostic procedure used to investigate lumps or masses found in a patient. FNA fluid and tissue samples can be taken from many parts of the body including, but not limited to, lungs, liver, thyroid, breast, lymph nodes, kidneys, and pancreas. In this technique, after locating a mass for biopsy, a puncture may be created using an elongate hollow needle 108, from the elongate hollow needle assembly 102. As shown in FIG. 1A, in certain embodiments, the hollow needle assembly 102 may be a longitudinal body that extends from an open distal end 114 to an open proximal end 116 and may include an elongate hollow needle 108, a hub 110, and a hollow tube 112. In other embodiments, the elongate hollow needle assembly may only include the elongate hollow needle 108. The elongate hollow needle 108 may be formed of any suitable biocompatible material as would be understood by those skilled in the art and include a puncturing tip 118 at the distal end 114. In some examples, a needle gauge for the elongate hollow needle 108 may range between 14 G and 28 G, as is commonly employed to obtain histological samples. It is noted, however, that this is not a requirement and in some embodiments, the needle gauge may be larger or smaller. In some cases, the elongate hollow needle assembly 102 may be formed as a single-piece. However, in other cases, one or more of the elongate hollow needle components (e.g., the elongate hollow needle 108, the hub 110, and the hollow tube 112) may be coupled to one another using one or more pins, staples, threads, screws, helix, tines, and/or the like. In some cases, each of the components of the elongate hollow needle assembly 102 may have an exterior wall and an interior wall. As shown in FIG. 1B, when the components of the elongate hollow needle assembly 102 are coupled together, the exterior walls and interior walls may define a needle lumen or a hollow interior that traverses from a distal open end 114 to a proximal open end 116 of the elongate hollow needle assembly 102.

In some cases, the elongate hollow needle 108 may be passed into the mass, for example under visual, ultrasound, x-ray, and/or palpation guidance. In some cases, the elongate hollow needle 108 may be inserted and withdrawn several times. After the elongate hollow needle 108 is placed into the mass, fluid can be drawn from the elongate hollow needle assembly 102 into the collection device 104 using the suction assembly 106. As shown in FIG. 1B, in various embodiments, the collection device 104 may be in-line with the elongate hollow needle assembly 102. In some cases, the elongate hollow needle assembly 102 and the collection device 104 may be formed as a single-piece. In other cases, a distal end 132 of the collection device 104 may be coupled to the open proximal end 116 of the elongated hollow needle assembly 102 using one or more pins, staples, threads, screws, helix, tines, and/or the like. Regardless of whether the elongate hollow needle assembly 102 and the collection device 104 are one solid piece or coupled together, the collection device 104 may be in fluid communication with the elongate hollow needle assembly 102 and be used to separate the desired tissue samples from the fluid. In certain embodiments, the collection device 104 may contain a membrane filtration structure 128 inside an external casing 130 and the membrane filtration structure 128 can collect the tissue samples directly from the fluid.

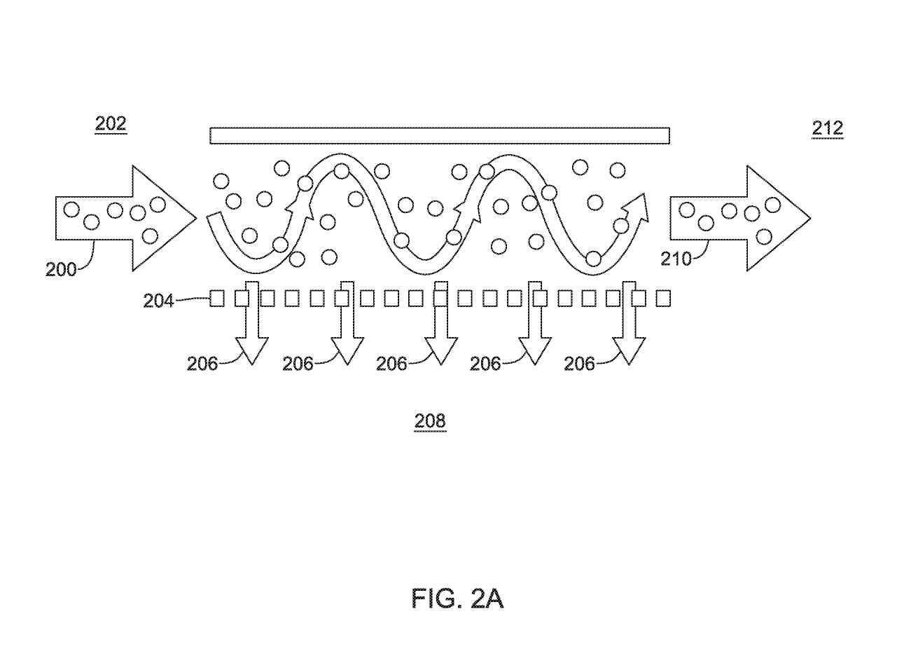

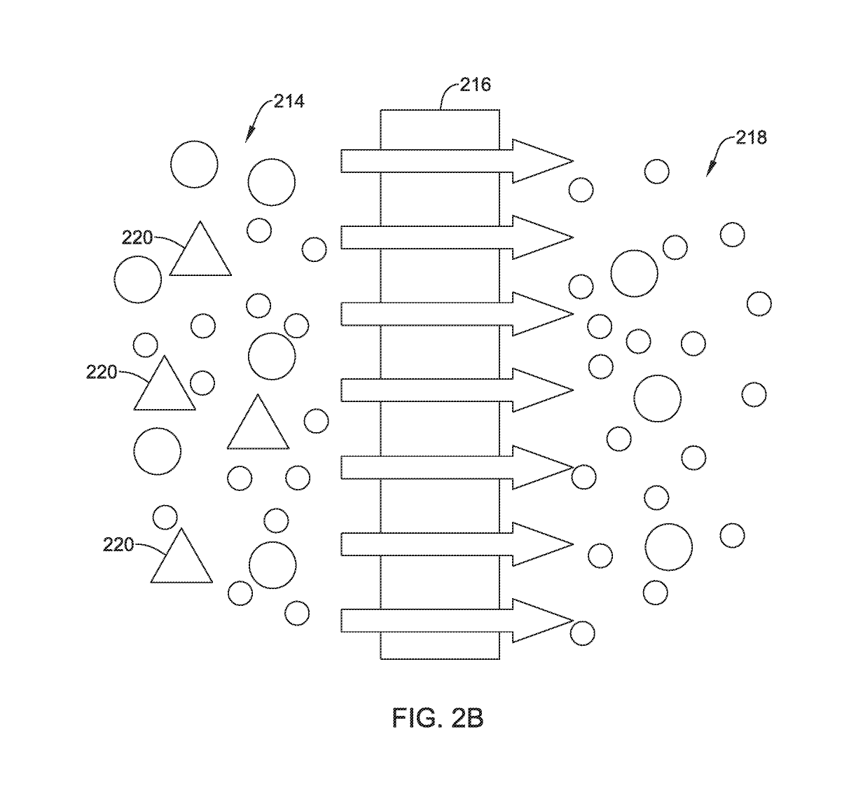

Membrane filtration can pertain to the use of permeable membranes to separate or filter substances. In some cases, the membrane may be structured for cross-flow or tangential flow filtration. Turning to FIG. 2A, an example of cross-flow filtration is depicted. As shown, in cross-flow filtration, a fluid 200 may flow on a feed side 202, parallel to a membrane surface 204. In certain examples, a proportion of the fluid (e.g., non-tissue 206) which are smaller than the membrane pore size may pass through the membrane as permeate 208 or filtrate and larger particles (e.g., tissue samples 210) may be retained on the feed side of the membrane as retentate 212.

In some cases, the membrane may be structured for dead-end filtration. Turning to FIG. 2B, an example of dead-end filtration is depicted. As shown, in dead-end filtration, a fluid 214 may flow perpendicular to a membrane surface 216, which may then allow passage of some particles (e.g., non-tissue 218) which are smaller than the membrane pore size and collect the larger particles (e.g., tissue samples 220).

Turning back to FIG. 1B, in various embodiments, the collection device 104 may also be classified according to the pore size of the membrane filtration structure 128. According to various embodiments, the pore size of the collection device 104 may be sized to filter cells around 0.1 to 10 μm in diameter.

In some cases, the collection device 104 may be a microfiltration system. In some examples, the pore size used for microfiltration may range from about 0.1 to 10 μm. In terms of molecular weight, these membranes may separate macromolecules of molecular weights generally less than 10̂8 g/mol. Microfiltration systems may be used to prevent particles such as, cells, for example, from passing through. More microscopic, atomic or ionic materials such as water (H₂O), monovalent species such as Sodium (Na⁺) or Chloride (Cl⁻) ions, dissolved or natural organic matter, and small colloids and viruses may still be able to pass through the microfiltration system. In some cases, the material of the membrane filter structure 128 used in microfiltration systems may be either organic or inorganic depending upon the contaminants that are desired to be removed, or the type of application. Organic membranes may be made using a diverse range of polymers including cellulose acetate (CA), for example. These may be used because of their flexibility, and chemical properties. Inorganic membranes may be composed of sintered metal or porous alumina. They may be designed in various shapes, with a range of average pore sizes and permeability.

In some cases, the collection device 104 may be an ultrafiltration system. In some examples, the pore size used for ultrafiltration may range from about 2 to 100 nm. In terms of molecular weight, these membranes may separate macromolecular solutions ranging from 10³ to 10⁶ Da, especially protein solutions. In some cases, the material of the membrane filter structure 128 used in ultrafiltration systems may comprise polymer materials such as, polysulfone, polypropylene, cellulose acetate, and polylactic acid, for example.

In some cases, the collection device 104 may be a nanofiltration system. Nanofiltration may be a membrane filtration structure 128 that uses nanometer sized cylindrical through-pores that may pass through the membrane at 90°. In some examples, the pore sized used for nanofiltration may range from about 1 to 2 nm, smaller than that used in microfiltration and ultrafiltration. Membranes used may be created from polymer thin films. Materials that may be used include, but are not limited to, polyethylene terephthalate or metals such as aluminum. Pore dimensions may be controlled by pH, temperature, and time during development with pore densities ranging from about 1 to 106 pores per cm². Membranes made from metal such as alumina membranes, may be made by electrochemically growing a thin layer of aluminum oxide from aluminum metal in an acidic medium. In some cases, nanofiltration systems may be used to prevent amino acids, lipids, and other cell cultures in the blood from passing through.

The above list of filtration systems is by no means exhaustive. In some cases, the membrane filtration structure 128 may have other configurations that facilitate separating of the desired tissue samples from the fluid. For example, the membrane filtration structure 128 of the collection device 104 may be configured for reverse osmosis, electrolysis, dialysis, electrodialysis, gas separation, vapor permeation, pervaporation, membrane distillation, membrane contactors, or combinations thereof. As such, the final design may be optimized for a multiple of factors including size of the separated particles, cost, ease of use, for example.

Alternatively or additionally, in certain embodiments, the membrane filtration structure 128 may be comprised of a/nitrocellulose/collodion membrane. In some cases, the porous character of the nitrocellulose/collodion membrane may be selectively permeable to cations. As such, the nitrocellulose/collodion membrane may allow the cations of univalent strong inorganic electrolytes in solutions to pass through, whereas the nitrocellulose/collodion membrane may be almost impermeable to anions. This ionic selectivity may be due to the negative electrical charge of the nitrocellulose/collodion membrane arising from the presence of dissociable acidic groups on the pore walls of the nitrocellulose/collodion membrane.

As stated above, according to various embodiments, the collection device 104 may have an exterior casing 130 substantially surrounding the membrane filtration structure 128. The material used to make the exterior casing 130 may be any suitable material that may be a pliable translucent or transparent material (e.g., polylactic acid, straw fibers, glycerol, poly-diphenyl methane diisocyanate, nisin, methylene chloride, and natamycin). The exterior casing 130 may also include the distal end 132 and a proximal end 134. As shown in FIG. 1B, the proximal end 134 may be configured to couple to a distal end 136 of the suction assembly 106 using one or more pins, staples, threads, screws, helix, tines, and/or the like or any other coupling device capable of establishing fluid communication of the tissue filtration device 100. In other embodiments, the collection device 104 and the suction assembly 106 may be formed as a single-piece. As shown in FIG. 1B, the collection device 104 and the suctions assembly 106 may be co-axial or in-line with one another.

As stated above, the fluid can be drawn from the elongate hollow needle assembly 102 into the collection device 104 using the suction assembly 106. In some cases, the suction assembly 106 may operate as a pump to move the fluid from the elongate hollow needle assembly 102 into the collection device 104. As shown in FIGS. 1A-1B, in some embodiments, the suction assembly 106 may be a syringe. A syringe may be a reciprocating pump consisting of a piston 120 that fits within a cylindrical tube 122. In some examples, the piston 120 can be linearly pulled and pushed along the inside of the cylindrical tube 122, allowing the tissue filtration device 100 to take in fluid from the distal open end 114 of the elongate hollow needle 108 to the collection device 104 and expel fluid from the collection device 104 through the distal open end 114.

Although a syringe is depicted in FIGS. 1A-1B, any suitable suction assembly 106 that creates a suction or pump that carries the fluid from the elongate hollow needle assembly 102 to the collection 104 may be used. For example, the suction assembly 106 may be powered via many energy sources such as manual operation, electricity, engines, etc. Furthermore, the type of pump of the suction assembly 106 may include, but is not limited to, positive displacement pumps, rotary positive displacement pumps, reciprocating positive displacement pumps, rotary lobe pumps, progressive cavity pumps, rotary gear pumps, piston pumps, diaphragm pumps, screw pumps, gear pumps, hydraulic pumps, rotary vane pumps, peristaltic pumps, rope pumps, flexible impellers, triplex-style plunger pumps, compressed-air-powered double-diaphragm pumps, impulse pumps, and velocity pumps.

Once proper tissue samples have been aspirated from the patient using the tissue filtration device 100, the collection device 104 may be removed from the tissue filtration device 100 and the tissue samples may be processed. In some cases, the tissue samples may be put on slides and stained. For instance, the tissue samples may be smeared from the collection device 104 directly onto the slide. In another example, the tissue samples may be put through a tissue processing procedure, embedded in a paraffin cell block, cut and stained. In another example, the tissue samples may be put through a tissue processing procedure and then scraped off the collection device 104 before embedding the tissue samples in a paraffin cell block. In some cases, the collection device 104 may be submitted for tissue processing and encased in a paraffin cell block. The advantages of encasing the entire collection device 104 in paraffin and putting slices of the paraffin block on slides may be that more slides can be made, allowing for immunohistochemical staining and molecular diagnostics, and that the tissue samples can be stored in the block for years. Once processed, the tissue samples may then be examined. The tissue samples are generally examined under a microscope by a pathologist, and can also be analyzed chemically, for example.

Referring now to FIG. 1C, an exemplary method for aspirating fluid from a patient and separating tissue samples 138 from the fluid using the tissue filtration device 100 will now be described. As shown, the exemplary method may be performed on a pancreas 126 of the patient. With the widespread and increasing use of high-resolution abdominal cross-sectional imaging, more and more pancreatic cystic lesions (PCLs) may be detected. Patients with a PCL may or may not have symptoms arising from the lesion, which may be completely benign without any malignant potential, may be benign but could become malignant, or already may be malignant. Visual guided pancreatic fluid sampling may be done using an endoscope 124 to traverse a digestive lumen (not shown), through a pancreatic duct 144, and to a branch duct 146. In certain embodiments, the endoscope 124 may include a visual camera 148 used to display images of the inside of the patient (e.g., the digestive lumen, the pancreatic duct 144, and the branch duct 146) to help an operator navigate the endoscope 124 to a target tissue 142. In various embodiments, the elongate hollow needle 108 of the elongate hollow needle assembly 102 may be positioned and guided through a working channel 140 of the endoscope 124. In some cases, the elongate hollow needle 108 may be a 19 to 27 gauge needle with an attached 2 to 20-mL suction assembly 106. In some cases, the needle gauge may be chosen based on the potentially high viscosity of fluid content in case of mucin rich lesions. Other factors to consider when decided on the needle gauge may be the location of target tissue 142 and the distance of the needle passage, for example. In some examples, a syringe holder (not shown) may or may not be used, according to the preference of the operator. Before aspiration, scanning may be performed on the branch duct 146 for target tissue 142 localization, followed by color Doppler mapping to depict any large blood vessels in and around the target tissue 142. The puncturing tip 118 of the elongate hollow needle 108 may then be introduced at or near the target tissue 142. When the elongate hollow needle 108 reaches the target tissue 142, fluid from the target tissue 142 may be moved to the collection device 104 using the reciprocating pump (e.g., syringe) suction assembly 106. During the procedure, all needle movements should be continuously visualized in real time (e.g., using visual imaging from the visual camera 148, using ultra-sound visualization, etc.). In this embodiment, the membrane filtration structure 128 of the collection device 104 may be a nitrocellulose/collodion membrane filtration structure 128 configured for cross-flow filtration. As such, the fluid flowing into the collection device 104 may flow on the feed side, parallel to the nitrocellulose/collodion membrane surface. Accordingly, a proportion of the fluid (e.g., non-tissue) which are smaller than the nitrocellulose/collodion membrane pore size may pass through the nitrocellulose/collodion membrane as permeate or filtrate and the larger particles (e.g., the tissue samples 138) may be retained on the feed side of the nitrocellulose/collodion membrane as retentate. In some cases, the following procedure may be repeated to ensure adequate tissue samples 138 have been obtained. Once aspiration is complete, the elongate hollow needle 108 may be removed from the pancreas 126 and the patient. The collection device 104 may then be removed from the tissue filtration device 100 and the feed side of the nitrocellulose/collodion membrane filtration structure 128 containing the tissue samples 138 may be processed and examined.

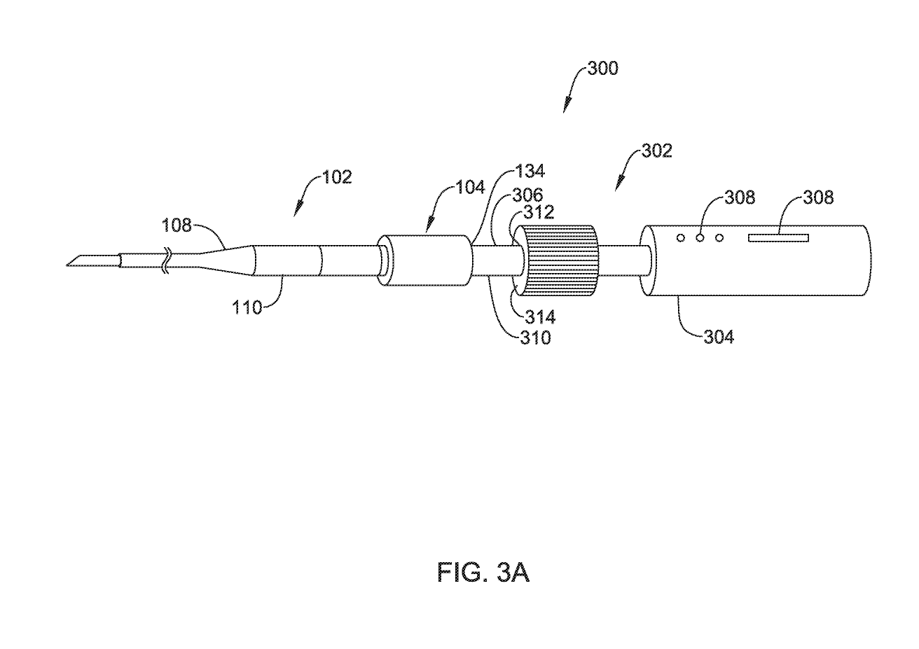

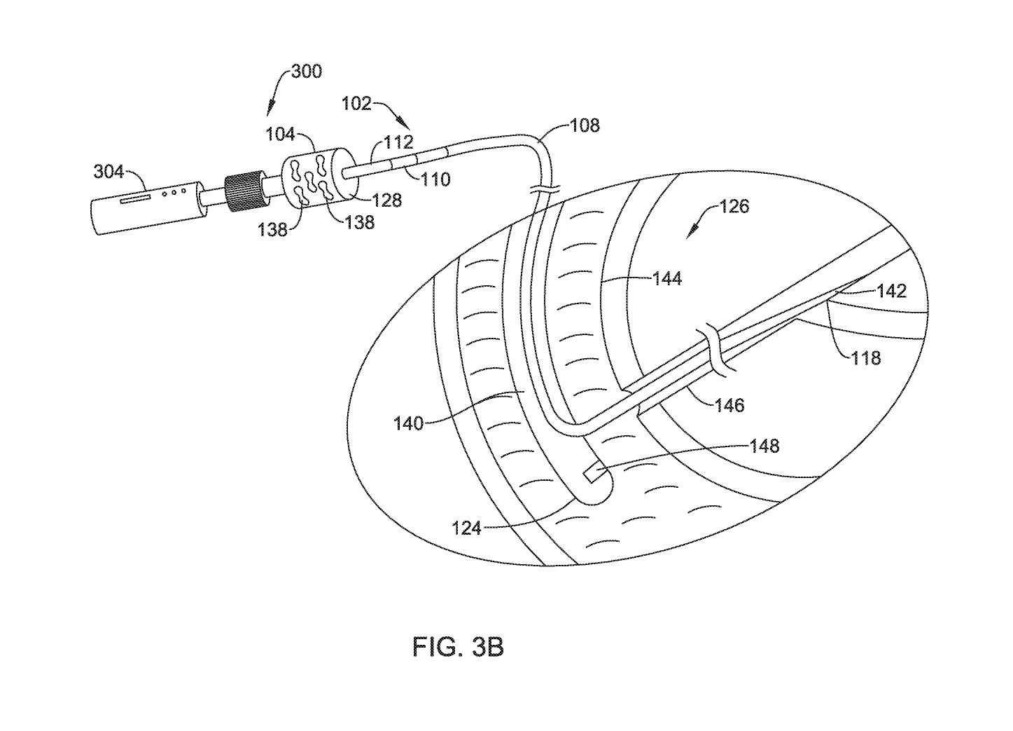

FIG. 3A illustrates an example of a second tissue filtration device 300 that may be used to aspirate fluid from a patient and separate tissue samples from the fluid. According to various embodiments, the tissue filtration device 300 may include the elongate hollow needle assembly 102, the collection device 104, and a suction assembly 302. The configuration and operation of the second tissue filtration device 300 may be similar to the configuration and operation of the tissue filtration device 100 described with respect to FIGS. 1A-1C. However, the suction assembly 302 may include a handle assembly 304 and a hollow tube 306. In some cases, a distal end 310 of the hollow tube 306 may be coupled to the proximal end 134 of collection device 104 and a proximal end 312 of the hollow tube 306 may be coupled to a proximal end 314 of the handle assembly 304. In certain embodiments, the handle assembly 304 may include a mechanism 308. In some cases, the mechanism 308 may be actuated to activate a pump inside the handle assembly 304 to move the fluid from the elongate hollow needle assembly 102 into the collection device 104. For example, an operator (e.g., a physician) may press the mechanism 208 that activates a positive displacement pump within the handle assemble 304, creating a suction that draws the fluid from the patient, into the elongate hollow needle assembly 102 and into the collection device 104. Furthermore, as shown in FIG. 2A, in certain embodiments, the hollow tube 112 may be absent from the elongate hollow needle assembly 102.

Referring now to FIG. 3B, an exemplary method for aspirating fluid from a patient and separating tissue samples 138 from the fluid using the second tissue filtration device 300 will now be described. Similar to the exemplary method performed by the tissue filtration device 100 and described with respect to FIG. 1C, the exemplary method performed by the second tissue filtration device 300 may also be done on the pancreas 126. In various embodiments, the endoscope 124 may traverse the digestive lumen (not shown), through the pancreatic duct 144, and to the branch duct 146. In certain embodiments, the endoscope 124 may include the visual camera 148 used to display images of the inside of the patient (e.g., the digestive lumen, the pancreatic duct 144, and the branch duct 146) to help an operator navigate the endoscope 124 to a target tissue 142. In some cases, the elongate hollow needle 108 of the elongate hollow needle assembly 102 may be positioned and guided through a working channel 140 of the endoscope 124. The puncturing tip 118 of the elongate hollow needle 108 may be introduced into the patient at or near the target tissue 142. When the elongate hollow needle 108 reaches the target tissue 142, fluid may be moved into the collection device 104 by actuating the mechanism 308 activating a positive displacement pump within the handle assemble 304, creating a suction that draws the fluid from the patient, into the elongate hollow needle assembly 102 and into the collection device 104. In this embodiment, the membrane filtration structure 128 of the collection device 104 may once again be the nitrocellulose/collodion membrane filtration structure 128 configured for cross-flow filtration. As such, the fluid flowing into the collection device 104 may flow on the feed side, parallel to the nitrocellulose/collodion membrane surface. Accordingly, a proportion of the fluid (e.g., non-tissue) which are smaller than the nitrocellulose/collodion membrane pore size may pass through the nitrocellulose/collodion membrane and the larger particles (e.g., the tissue samples 138) may be retained on the feed side of the nitrocellulose/collodion membrane. Once aspiration is complete, the elongate hollow needle 108 may be removed from the pancreas 126 and the patient. The collection device 104 may then be removed from the tissue filtration device 300 and the feed side of the nitrocellulose/collodion membrane filtration structure 128 containing the tissue samples 138 may be processed and examined.

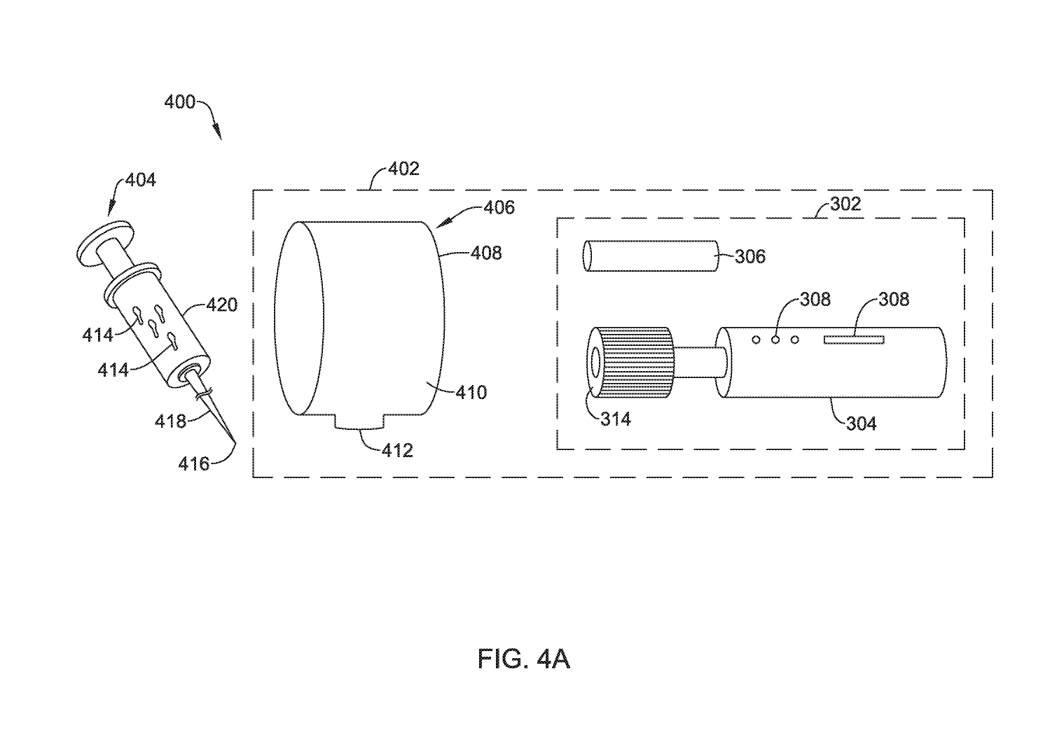

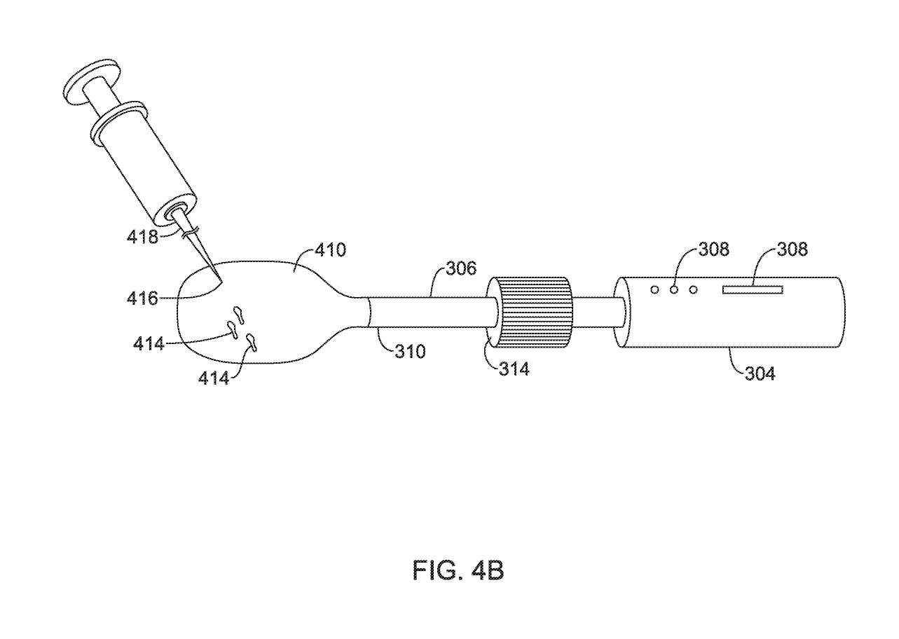

FIG. 4A illustrates an example of a tissue filtration system 400 that may be used to aspirate fluid from a patient and separate tissue samples from the fluid. According to various embodiments, the tissue filtration system 400 may include a tissue filtration device 402 and a biopsy needle assembly 404. The biopsy needle assembly 404 may be any suitable biopsy needle assembly known and understood by those skilled in the art. In certain embodiments, the tissue filtration device 402 may include the suction assembly 302 and a collection device 406. The collection device 406 may be configured and operate similar to the collection device 104, from FIGS. 1A-1C. In certain embodiments, the collection device 406 may include an exterior casing 408 and a membrane filtration structure 410. In some cases, the exterior casing 408 may be configured to maintain an airtight seal when punctured by the biopsy needle 404. The material used to fabricate the exterior casing 408 may be any suitable material that can be punctured by the biopsy needle assembly 404 and maintain an airtight seal. These materials may be a pliable translucent or transparent material. The exterior casing 408 may also include an opening 412 that may be configured to couple to the suction assembly 302. The opening 412 may be coupled to the suction assembly using one or more pins, staples, threads, screws, helix, tines, and/or the like or any other coupling device capable of creating an air tight seal between the opening 412 and the suction assembly 302.

FIG. 4B illustrates an example of the tissue filtration system 400 in operation. As shown, the distal end 310 of the hollow tube 306 is coupled to the opening 412, creating an air tight seal between the opening 412 and the suction assembly 302. In other embodiments, the hollow tube 306 may be absent from the suction assembly 302 and the opening 412 may be coupled to the distal end 314 of the handle assembly 304, creating an air tight seal between the opening 412 and the suction assembly 302. In certain embodiments, a puncturing tip 416 at a proximal end 418 of the biopsy needle assembly 404 may be introduced to the collection device 406. As stated above, when the collection device 406 is punctured by the biopsy needle assembly 404, the exterior casing 408 may be configured to maintain an airtight seal such that neither air nor the membrane filtration structure 410 may seep out of the exterior casing 408. In some cases, a handle assembly 420 may be coupled to the proximal end 418 of the biopsy needle assembly 404 and contain the fluid. In certain embodiments, the fluid may be moved to the collection device 406 by actuating the mechanism 308 activating a positive displacement pump within the handle assemble 304, creating a suction that draws the fluid from the handle assembly 420, into the proximal end 418 and into the collection device 406. In this embodiment, the collection device 406 may be configured for cross-flow filtration. As such, the fluid flowing into the collection device 406 may flow on the feed side, parallel to a membrane surface of the membrane filtration structure 410. Accordingly, a proportion of the fluid (e.g., non-tissue) which are smaller than the membrane filtration structure 410 pore size may pass through the membrane filtration structure 410 and the larger particles (e.g., the tissue samples 414) may be retained on the feed side of the membrane filtration structure 410. Once movement of the fluid is complete, the puncturing tip 416 may be removed from the collection device 406. In various embodiments, the exterior casing 408 may be configured to maintain an airtight seal once the puncturing tip 416 is removed such that air may not enter the collection device 406 and the membrane filtration structure 410 nor the tissue samples 414 may seep out of the exterior casing 408. The collection device 406 may then be disconnected from the suction assembly 302 and the feed side of the membrane filtration structure 410 containing the tissue samples 414 may be processed and examined.

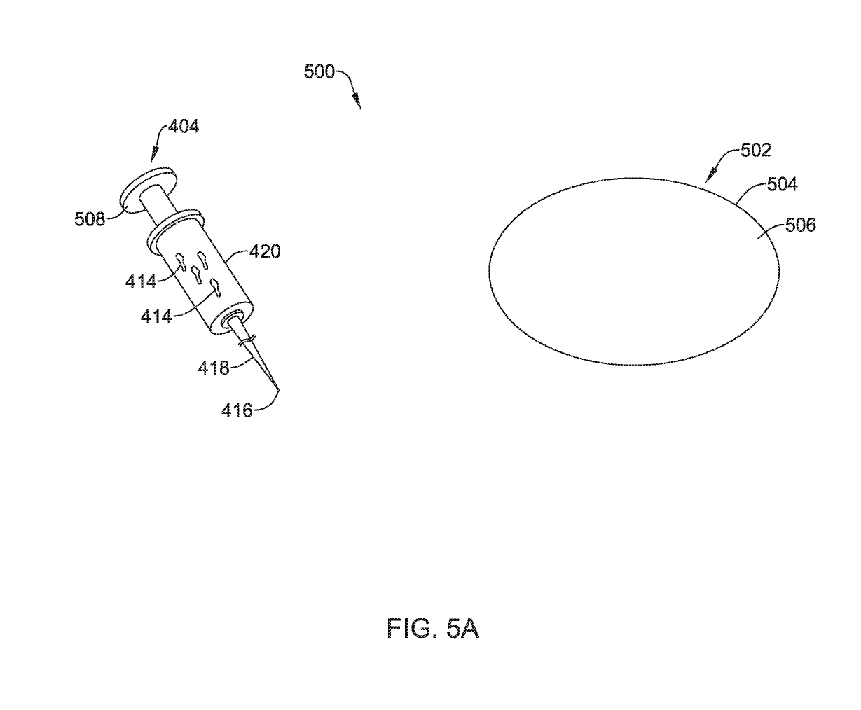

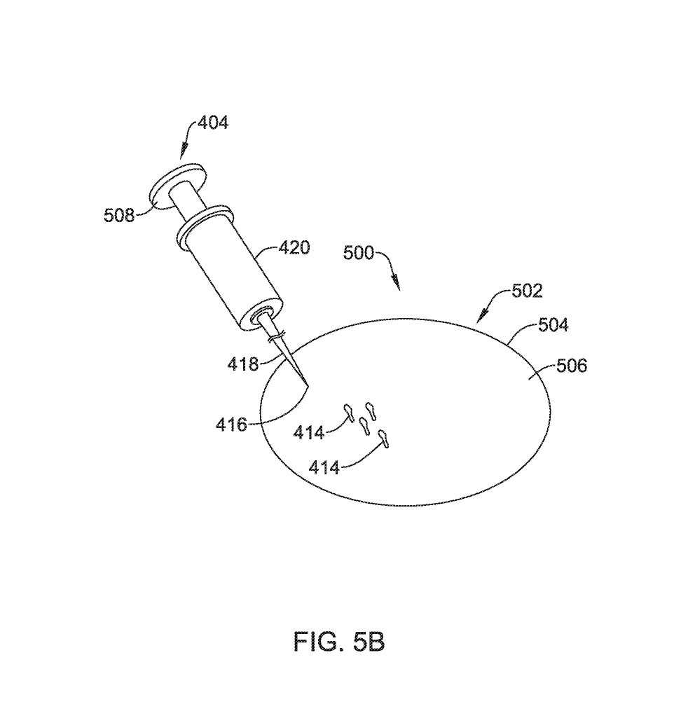

FIG. 5A illustrates an example of a second tissue filtration system 500 that may be used to aspirate fluid from a patient and separate tissue samples 414 from the fluid. According to various embodiments, the tissue filtration system 500 may include a collection device 502 and the biopsy needle assembly 404. In certain embodiments, the collection device 502 may be configured and operate similar to the collection device 406. As shown, the collection device 502 may include an exterior casing 504 and a membrane filtration structure 506. In some cases, the exterior casing 504 may be configured to maintain an airtight seal when punctured by the biopsy needle 404. The material used to fabricate the exterior casing 504 may be any suitable material that can be punctured by the biopsy needle assembly 404 and maintain an airtight seal. These materials may be a pliable translucent or transparent material.

FIG. 5B illustrates an example of the tissue filtration system 500 in operation. As shown, in certain embodiments, the puncturing tip 416 of the biopsy needle assembly 404 may be introduced to the collection device 502. As stated above, when the collection device 502 is punctured by the biopsy needle assembly 404, the exterior casing 504 may be configured to maintain an airtight seal such that neither air nor the membrane filtration structure 506 may seep out of the exterior casing 504. In some cases, the handle assembly 420 may be coupled to the proximal end 418 of the biopsy needle assembly 404 and contain the fluid. In certain embodiments, the fluid may be moved to the collection device 502 using a reciprocating pump 508 (e.g., syringe) of the handle assembly 420 that pushes the fluid into the proximal end 418 and into the collection device 502. In this embodiment, the collection device 502 may be configured for cross-flow filtration. As such, the fluid flowing into the collection device 502 may flow on the feed side, parallel to a membrane surface of the membrane filtration structure 506. Accordingly, a proportion of the fluid (e.g., non-tissue) which are smaller than the membrane filtration structure 506 pore size may pass through the membrane filtration structure 506 and the larger particles (e.g., the tissue samples 414) may be retained on the feed side of the membrane filtration structure 506. Once movement of the fluid is complete, the puncturing tip 416 may be removed from the collection device 502. In various embodiments, the exterior casing 504 may be configured to maintain an airtight seal once the puncturing tip 416 is removed such that air may not enter the collection device 502 and the membrane filtration structure 506 nor the tissue samples 414 may seep out of the exterior casing 504. The feed side of the membrane filtration structure 506 containing the tissue samples 314 may then be processed and examined.

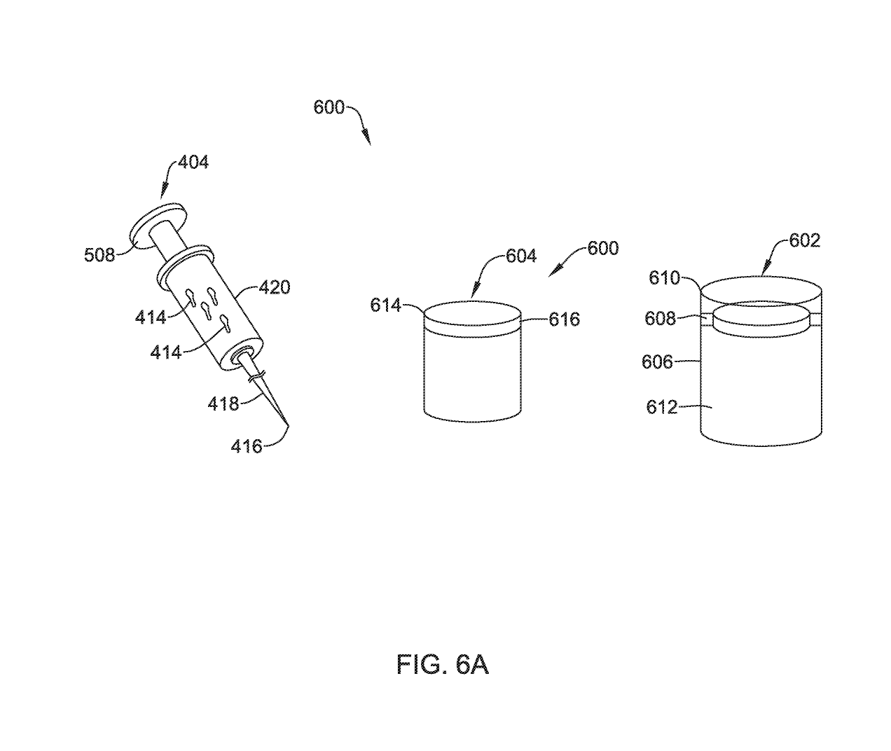

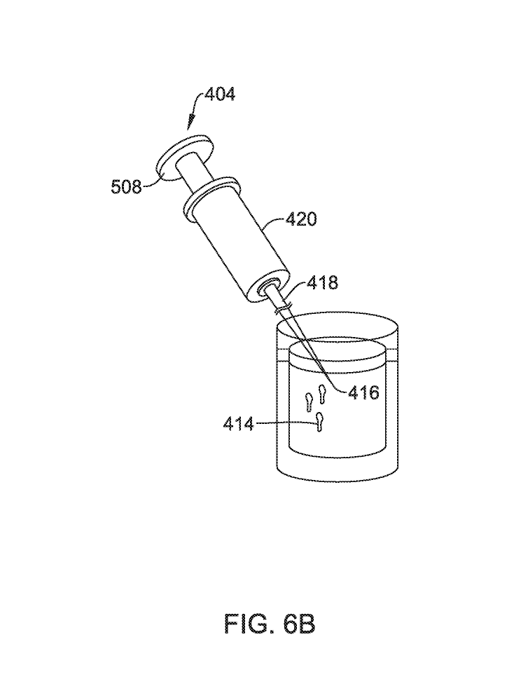

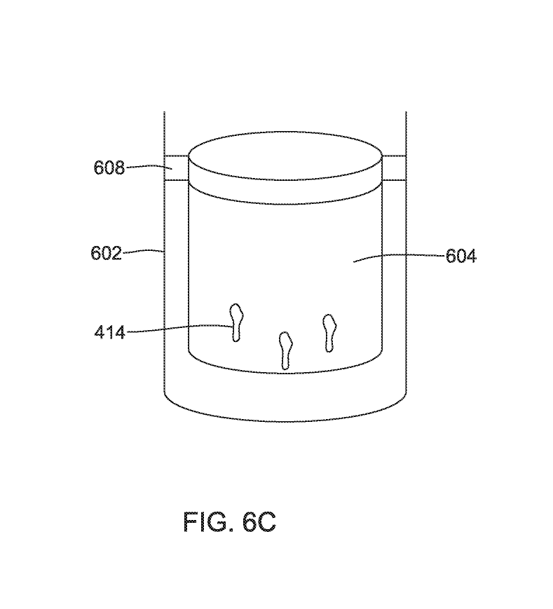

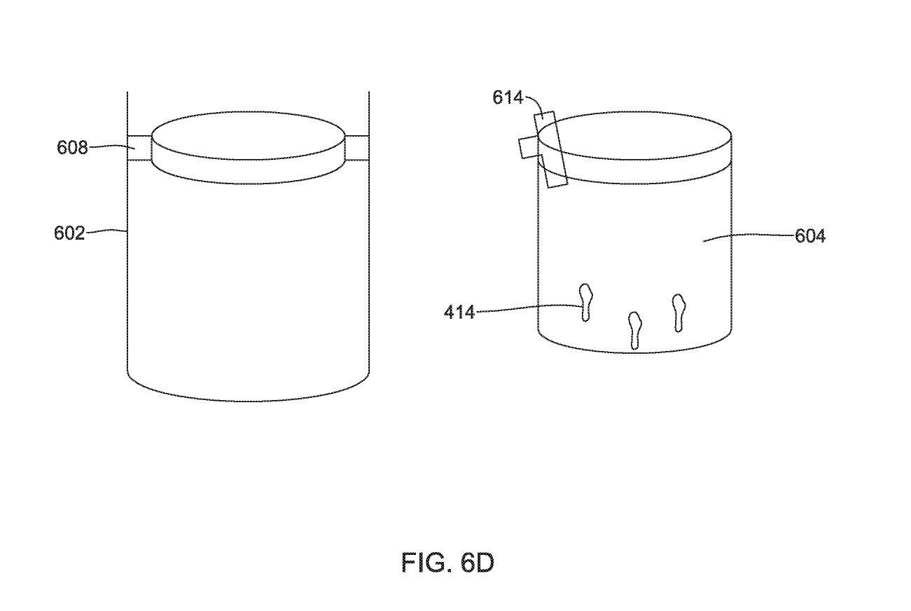

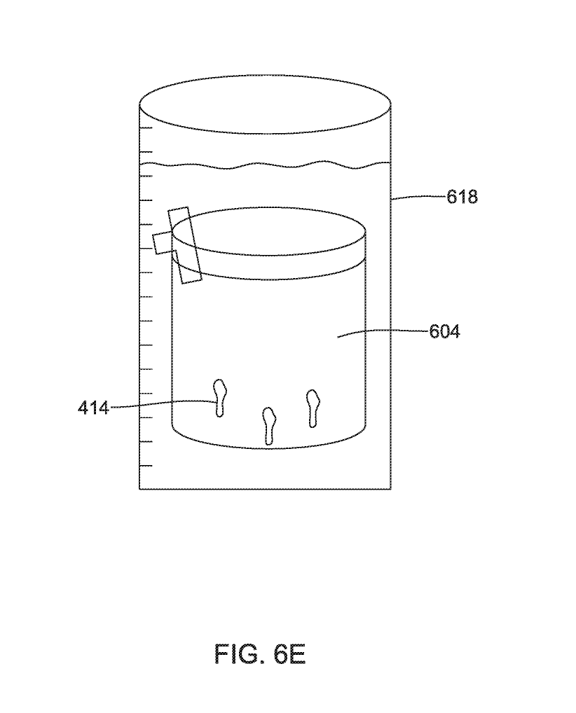

FIGS. 6A-6E illustrate an example method of a third tissue filtration system 600 that may be used to aspirate fluid from a patient and separate tissue samples 414 from the fluid. According to various embodiments, the tissue filtration system 600 may include the biopsy needle assembly 404, a sample container 602, and a filtered sample bag 604.

As shown in FIG. 6A, the sample container 602 may include a housing 606, and a rack 608 attached near an open end 610 of the housing 606. In this embodiment, the housing 606 may be cylindrical in shape and include an interior region 612 into which the sample bag 604 may extend. The housing 606 and the rack 608 may be fabricated from material such as a polycarbonate material, or other materials such as polyetheretherketone, chlorotrifluoroethylene, or borosilicate glass, for example. In some cases, the housing 606 and the rack 608 may be formed as a single-piece. However, in other cases, the housing 606 and the rack may be coupled to one another using one or more pins, staples, threads, screws, helix, tines, and/or the like. The filtered sample bag 604 may be fabricated from material such as cellulose, polytetrafluoroethylene, polyvinylidene fluoride, other materials, and the like. In this embodiment, the filtered sample bag 604 may be shaped and sized to contain milliliters of samples. However, the filtered sample bag 604 may assume other shapes and sizes in alternative embodiments. In some embodiments, the filtered sample bag 604 may be configured with a sealing structure 614 at an open end 616 of the filtered sample bag 604. The sealing structure 614 may be any suitable sealing structure known and understood by those skilled in the art, such as a zip-lock, a cinch, pins, hoops, hooks, and tines, for example. In some embodiments, the filtered sample bag 604 and the sealing structure 614 may be composed of dissimilar materials. In other embodiments, the filtered sample bag 604 and the sealing structure 614 may be composed of the same materials. In some cases, the filtered sample bag 604 and the sealing structure 614 may be attached through a molding process or using an adhesion process, for example.

Turning to FIG. 6B, in some examples, the filtered sample bag 604 may be placed through and on the rack 608 of the sample container 602. In some embodiments, the filtered sample bag 604 may be secured to the rack 608 using one or more pins, staples, threads, screws, helix, tines, hooks, and/or the like. In other embodiments, the filtered sample bag 604 may simply hang from the rack 608. In some cases, the puncturing tip 416 of the biopsy needle assembly 404 may then be introduced at the open end 616 of the sample bag 604. In some cases, the handle assembly 420 may be coupled to the proximal end 418 of the biopsy needle assembly 404 and contain the fluid. In certain embodiments, the fluid may be moved to the sample bag 604 using the reciprocating pump 508 (e.g., syringe) of the handle assembly 420 that pushes the fluid into the proximal end 418 and into the filtered sample bag 604. As shown in FIG. 6C, once the fluid has been moved into the sample bag 604, the puncturing tip 416 may be removed from the open end 616 of the filtered sample bag 604 and the filtered sample bag 604 may be removed from the rack 608 of the sample container 602. As shown in FIG. 6D, the sealing structure 614 may then be moved to close and/or seal the filtered sample bag 604. As shown in FIG. 6E, the sample bag 604 may then be transferred into a formalin-filled holding vessel 618. When the tissue samples 414 are ready for processing, the filtered sample bag 604 may be removed from the formalin-filled holding vessel 618 and transferred to a processing cassette for histological processing.

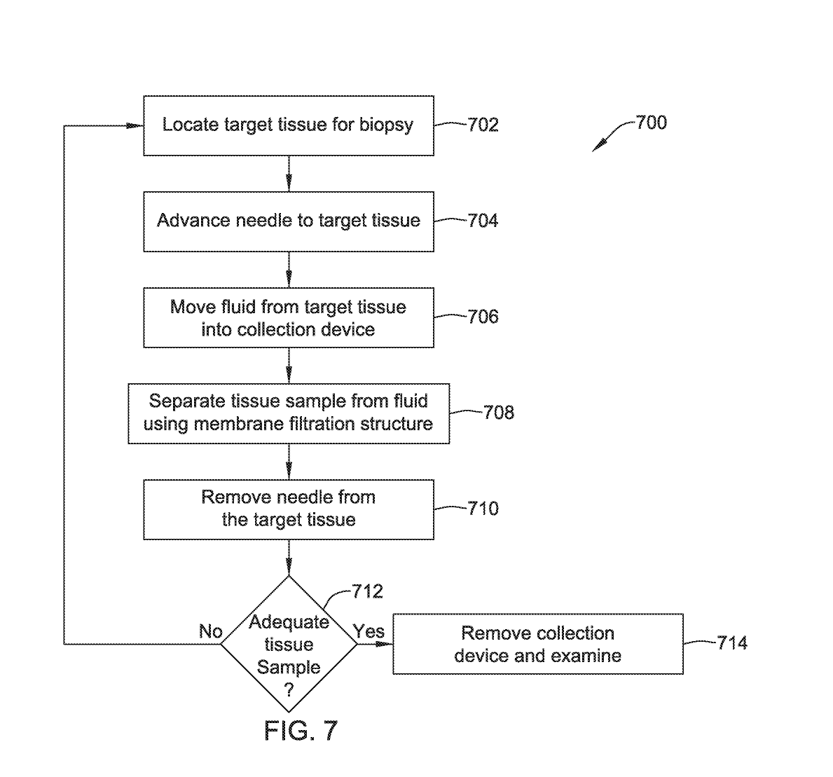

FIG. 7 depicts an illustrative flow-diagram method 700 for aspirating fluid from a patient and separating tissue samples from the fluid using an exemplary tissue filtration device (e.g., tissue filtration devices 100 and 300). The method 700 may begin at step 702 where target tissue or a mass for biopsy may be located in the patient. In some cases, an endoscope may be used to traverse the intestinal tract of the patient to the pancreas, where the target tissue is located. In some examples, scanning may be performed to locate the target tissue, followed by color Doppler mapping to depict any large blood vessels in and around the target tissue. At step 704, a needle may be advanced to the target tissue. In some cases, the needle may be located in a working channel of the endoscope. At step 706, when the needle has reached the target tissue, fluid from the target tissue may be moved to a collection device using a suction assemble of the tissue filtration device. At step 708, the membrane filtration structure of the collection device may separate the tissue samples from the fluid. In some examples, the membrane filtration structure may be configured for cross-flow filtration. In other examples, the membrane filtration structure may be configured for dead-end filtration. Furthermore, in some examples, the membrane filtration structure may be configured for microfiltration, ultrafiltration, nanofiltration, reverse osmosis, electrolysis, dialysis, electrodialysis, gas separation, vapor permeation, pervaporation, membrane distillation, membrane contactors, or combinations thereof. Alternatively or additionally, in some examples, the membrane filtration structure may be comprised of a nitrocellulose/collodion membrane. At step 710, the needle may be removed from the target tissue. At step 712, it may be determined whether adequate tissue samples have been obtained. If it is determined that adequate tissue samples have not been obtained, the method may move back to step 702 and steps 702 through 710 may be repeated. If it is determined that adequate tissue samples have been obtained, at step 714, the tissue filtration device may be removed from the patient, the collection device may be removed from the tissue filtration device, and the membrane filtration structure containing the tissue samples may be processed and examined.

What is claimed is:

1. A tissue filtration device, comprising:
a distal elongate hollow needle comprising a needle lumen with distal and proximal ends;
a collection device fluidly coupled to the needle lumen comprising a filter material; and
a proximal assembly configured to draw fluid from the needle lumen into the collection device.

2. The tissue filtration device of claim 1, wherein the distal elongate hollow needle includes an elongate hollow needle and a hollow tube proximal of the elongate hollow needle.

3. The tissue filtration device of claim 1, wherein the proximal assembly includes a handle and a hollow tube distal of the handle.

4. The tissue filtration device of claim 3, wherein the handle includes a piston inside a cylindrical tube and the fluid is drawn from the needle lumen of the distal elongate hollow needle to the collection device by moving the piston through the cylindrical tube.

5. The tissue filtration device of claim 3, wherein the handle includes a mechanism and the fluid is drawn from the needle lumen of the distal elongate hollow needle to the collection device by actuating the mechanism.

6. The tissue filtration device of claim 1, wherein the filter material filters tissue from the fluid.

7. The tissue filtration device of claim 1, wherein the filter material includes a filter membrane.

8. The tissue filtration device of claim 1, wherein the collection device comprises an exterior casing configured to maintain an airtight seal when punctured by the distal elongate hollow needle.

9. The tissue filtration device of claim 8, wherein the exterior casing comprises a plastic.

10. The tissue filtration device of claim 1, wherein the filter material filters cells over about 0.1-10 μm in diameter.

11. A fine needle aspiration (FNA) device, comprising:
a distal elongate hollow needle comprising a needle lumen with distal and proximal ends;
a collection device fluidly coupled to the needle lumen and containing a filter material; and
a proximal handle configured to draw fluid from the needle into the collection device.

12. The FNA device of claim 11, wherein the collection device is coupled to the distal elongate hollow needle using a connecting mechanism.

13. The FNA device of claim 11, wherein the proximal handle is coupled to the collection device using a connecting mechanism.

14. The FNA device of claim 11, wherein the distal elongate hollow needle includes an elongate hollow needle and a hollow tube proximal of the elongate hollow needle.

15. The FNA device of claim 11, wherein the proximal handle includes a handle and a hollow tube distal of the handle.

16. The FNA device of claim 15, wherein the handle includes a piston inside a cylindrical tube and the fluid is drawn from the needle lumen into the collection device by moving the piston through the cylindrical tube.

17. The FNA device of claim 15, wherein the handle includes a mechanism and the fluid is drawn from the needle lumen into the collection device by actuating the mechanism.

18. The FNA device of claim 11, wherein the filter material filters tissue from the fluid.

19. The FNA device of claim 11, wherein the filter material includes a filter membrane.

20. A collection device configured to receive fluid from a biopsy needle, the collection device comprising:
an exterior casing configured to maintain an airtight seal when punctured by the biopsy needle;
a filter membrane enclosed by the exterior casing and configured to filter tissue from the fluid; and
an opening configured to couple the collection device to a suction assembly enabling the suction assembly to draw the fluid from the biopsy needle into the collection device.
